(19) United States
(12) Patent Application Publication
(10) Pub. No.: US 2016/0106797 A1
(43) Pub. Date: Apr. 21, 2016

(54) TOPICAL TREATMENT OF SHINGLES

(71) Applicant: Lou Paradise, Rhinebeck, NY (US)

(72) Inventor: Lou Paradise, Rhinebeck, NY (US)

(21) Appl. No.: 14/881,636

(22) Filed: Oct. 13, 2015

Related U.S. Application Data
(60) Provisional application No. 62/063,977, filed on Oct. 15, 2014.

(51) Int. Cl. A61K 36/81; A61K 36/22; A61K 36/77; A61K 41/00; A61L 15/44; A61L 15/42; A61L 15/40; A61K 36/28; A61K 9/00

(52) U.S. Cl. CPC A61K 36/81; A61K 36/28; A61K 36/22; A61K 36/77; A61K 9/0014; A61L 15/44; A61L 15/425; A61L 15/40; A61K 41/0004; A61L 2300/45

(57) ABSTRACT

The present invention relates to a homeopathic composition that may be used for treatment of chronic moderate to severe pain associated with virally driven neuropathies. More particularly, the present disclosure and claims provide a treatment of the pain symptoms of shingles, herpes zoster. In some embodiments, the application of the homeopathic composition may reduce length of the outbreak, severity of symptoms, and long term effects, such as nerve damage or skin discoloration.

### CROSS REFERENCE TO RELATED APPLICATIONS

This application claims priority to the United States Provisional application bearing the Ser. No. 62/063,977, filed Oct. 15, 2014 and entitled TOPICAL TREATMENT OF SHINGLES. The contents are relied upon and hereby incorporated by reference.

### FIELD OF INVENTION

The present invention relates to a homeopathic composition that may be used for treatment of chronic moderate to severe pain associated with virally driven neuropathies. More particularly, the present disclosure and claims provide a treatment of the pain symptoms of shingles, herpes zoster. In some embodiments, the application of the homeopathic composition may reduce length of the outbreak, severity of symptoms, and long term effects, such as nerve damage or skin discoloration.

### BACKGROUND

Approximately one million people in the United States are diagnosed with shingles, herpes zoster, each year. This viral disease is premised by a primary infection with varicella zoster (chicken pox) and is caused by a reactivated replication of a dormant virus that resides in ganglionic nerves.

The risk of shingles increases with age, and individuals with compromised immune systems may be particularly vulnerable. The disease comprises three phases: a pre-eruptive phase characterized by a multitude of sensory nerve phenomena, which may last 48-72 hours; an acute eruptive phase characterized by lesions, mild to severe pain, pruritus, and hyperesthesia, which may last 2-4 weeks; and a chronic phase characterized by post-herpetic neuralgia (PHN), which may persist 3-6 months or longer and may affect approximately 20% of shingles patients.

Currently available therapies include oral systemic medications, topical therapies, and vaccinations. However, the oral systemic medications primarily comprise non-FDA approved medications that may cause severe side effects. Vaccinations for shingles are in their infancy, and insufficient evidence exists regarding the long-term lasting effects on PHN pain.

The available topical therapies are often ineffective as monotherapy and may include general-use pain management solutions, which may not be particularly formulated to combat the symptoms associated with shingles. Current topical therapies have limited effectiveness, adverse side effects, and/or inconvenient application requirements. Generally, though, topical therapies have the least contraindications of all therapies currently available for PHN treatment, wherein topical therapies may be combined with systemic therapies without risk of adverse drug interactions.

Accordingly, what is needed therefore as a pain management therapy of all phases of shingles is a more effective and easily accessible topical treatment with fewer adverse side effects. There may be a particular need for pain management therapies during the extensive chronic phase.

### SUMMARY

Accordingly, the present invention provides a homeopathic composition that may be used for treatment of chronic moderate to severe pain associated with virally driven neuropathies, particularly shingles. In particular, the present invention provides a topical application, such as a cream or gel that may be applied on or into the skin, wherein the topical application includes one or more a vasodilator, stimulator of lymphatic activity, or mobilizers. Preferred embodiments include ingredients derived from natural sources, and in some aspects, natural sources processed through homeopathic steps. Homeopathic steps may include, for example, filtration and dilution.

One general aspect includes a method for treating shingles, the method including the steps of identifying a situs of a shingles condition on human skin. The method also includes applying a topical composition onto the situs of the shingles condition on human skin, where the composition includes an effective amount of a combination of herbs or extracts of arnica montana, rhus toxicodendron and aesculus hippocastanum mixed with an effective amount of belladona, in a pharmaceutically acceptable carrier to alleviate pain associated with the shingles condition. The method also includes the method where the topical composition includes a gel, a lotion or a cream.

Implementations may also include the method additionally including the steps of observing pain and fever in conjunction with the identification of the situs of the shingles condition and topically applying the topical composition onto the situs of shingles. The method may also include the method additionally including examples where the treatment bandage additionally include a second sealing layer for removably attaching the substrate and treating composition in a position proximate and in contact with the situs of shingles on the human skin. The method may additionally include the step of observing blisters on an area of skin in conjunction with the identification of the situs of the shingles condition and topically applying the topical composition onto the blisters. The method may additionally include the step of observing crusting on the blisters on the area of skin in conjunction with the identification of the situs of the shingles condition and topically applying the topical composition onto crusting blisters. The method may additionally include the step of applying a treatment bandage over the situs of shingles on the human skin where the treatment bandage includes a porous portion impregnated with the topical composition. The method additionally include the step of observing discoloration on an area of skin in conjunction with the identification of the situs of the shingles condition and topically applying the topical composition onto the area of discoloration.

One general aspect includes a treatment bandage for alleviating discomfort associated with shingles manifesting situs on human skin, the treatment bandage may include a first sealing layer formable to a contour of the situs manifesting shingles, and a substrate fixedly attached to the first sealing layer. The treatment bandage also includes a treating composition on a first surface of the substrate fixedly attached to the first sealing layer. In some examples, the treating composition includes an effective amount of a combination of herbs or extracts of arnica montana, rhus toxicodendron and aesculus hippocastanum mixed with an effective amount of belladona, in a pharmaceutically acceptable carrier.

Implementations may include one or more of the following features. The treatment bandage may additionally include a second sealing layer for removably attaching the substrate and treating composition in a position proximate and in contact with the situs of shingles on the human skin. The treatment bandage may also include abstract. The treatment bandage may also include the present invention

The details of one or more embodiments of the invention are set forth in the accompanying drawings and the description below. The accompanying drawings that are incorporated in and constitute a part of this specification, illustrate several embodiments of the invention and, together with the description, serve to explain the principles of the invention: Other features, objects, and advantages of the invention will be apparent from the description and the claims herein.

### BRIEF DESCRIPTION OF THE DRAWINGS

The accompanying drawings, that are incorporated in and constitute a part of this specification, illustrate several embodiments of the invention and, together with the description, serve to explain the principles of the invention:

FIG. 1A illustrates an anterior view of a human body, wherein common shingles sites are highlighted.

FIG. 1B illustrates a posterior view of a human body, wherein common shingles sites are highlighted.

FIG. 1C illustrates an exemplary progression of shingles symptoms on a cross section of skin with a subcutaneous nerve fiber.

FIG. 2A illustrates an exemplary outbreak of an infected user.

FIG. 2B illustrates an exemplary application of the homeopathic composition on an infected user.

FIG. 2C illustrates an alternate exemplary application of the homeopathic composition on an infected user.

FIG. 3A illustrates an exemplary outbreak of an infected user, wherein the affected site is facially located.

FIG. 3B illustrates an exemplary application of the homeopathic composition on the infected user.

FIG. 3C illustrates an exemplary treated user.

FIG. 4 illustrates an exemplary treatment bandage.

### DETAILED DESCRIPTION

The present invention relates generally to a method for treatment of chronic moderate to severe pain associated with virally driven neuropathies, such as, for example shingles. In particular, the present invention provides a topical application, such as a homeopathic cream or gel, which may be applied topically on or into the skin, wherein the topical application includes one or more a vasodilator, stimulator of lymphatic activity, or mobilizers. Preferred embodiments include ingredients derived from natural sources, and in some aspects, natural sources processed through homeopathic steps. Homeopathic steps may include, for example, filtration and dilution.

More specifically, the components of the composition may include a vasodilator, such as, for example, Arnica Montana, primrose oil, rosemary, ginger, Hamamelis, horse chestnuts, Yohimbe, niacin L-ornithate, and the like; a stimulator of lymphatic activity, such as, for example, Echinacea Augustofolia, St. Johns Wort, Belladonna, Devils Claw, Yellow Dock, Burdock, North American Ginseng, Wild Indigo, Pleurisy Root, and Pokeweed; and mobilizers of white blood cell activity, such as, for example, Lachesis Ninta venom, Crotalus horridus venom, Naja venom, Red Chinese Ginseng, Siberian Ginseng and Korean Ginseng, Goldenseal, Chamomile, and Barberry. Each of the components may be used in the amount of about 0.5 to 5%, preferably, 0.5 to 2.0% by weight.

In some embodiments, the homeopathic composition may include snake venom and venom extracts, which have anti-inflammatory capabilities. The snake venom may reduce swelling on or around skin lesions associated with shingles.

In some aspects, the homeopathic composition may include prostaglandins, which may have been found to improve nerve conduction velocity. In some embodiments, prostaglandins may be formed to be advantageous to induce prostaglandin production at the site affected by the disease rather than infusing the entire body. Accordingly, the inclusion of 0.5 to 5% by weight of the composition such as primrose oil, flax oil, and crocetin, may be beneficial.

In some embodiments, the homeopathic composition may include Caprylyl Glycol, which may function as a humectant and/or stabilizer. Caprylyl glycol may be plant derive or synthetic. Caprylyl glycol may increase antimicrobial activity of other preservatives, and may be used in conjunction with phenoxyethanol and chloroxylenol. The preservative blend may work on the skin's surface to give it a soft and smooth texture.

In some aspects, the homeopathic composition may include Caprylic/Capric Triglyceride, which may be produced by chemical reactions between various fatty acids and glycerol (synthetic glycerine). This largely synthetic mixed triester may be derived from coconut oil and glycerin and may include an emollient, dispersing agent, and solvent. In some aspects, Caprylic/Capric Triglyceride may include spreading properties that may facilitate gliding of the homeopathic composition on the skin.

In some embodiments, the homeopathic composition may include Caprylhydroxamic Acid, which may take place of traditional preservatives. Caprylhydroxamic Acid has the best length (C8) of C chain to promote degradation of cell membrane structures. Caprylhydroxamic Acid may maintain a unionized state in acid to neutral and may perform at a neutral pH. Caprylhydroxamic Acid may have excellent efficacy of fungistatic agents.

In some aspects, the homeopathic composition may include Centaurea Cyanus Flower Extract or cornflower extract. Centaurea Cyanus Flower Extract may include a skin conditioning agent and astringent that is rich in polyphenols, specifically flavonoids and anthocyanins. Cornflower extract may include anti-inflammatory properties, which may be effective on or around an affected area.

In some embodiments, the homeopathic composition may include Chelidonium Majus or swallow wort, which may commonly be used for treating warts. Swallow wort has appreciable tissue rejuvenating properties. The juice of Chelidonium majus has anti-bacterial/anti-fungal effect, which may be effective to slow or stop excessive bleeding.

In some aspects, the homeopathic composition may include Citrus Aurantium Amara (Bitter Orange) Flower Water may include flavonoids, which are anti-inflammatory, antibacterial, and antifungal. Orange flower water contains hydrophilic properties and may act as a natural surfactant and emulsifier to enhance the application of skin lotions and other cosmetics. In some aspects, the orange flower water may be safe to use directly on the skin, without requiring dilution, and may be used on blotchy, irritated skin. Orange flower water is smoothing, soothing, and brightening due to the skin regenerative and vitamin-rich properties of organic citrus flowers.

In some embodiments, the homeopathic composition may include Lauric Acid. Lauric Acid is a main fatty acid in coconut oil, which may be used as a moisturizer for the skin and may reduce redness and flaking of skin with minimal adverse side effects. In some aspects, lauric acid may facilitate healing of skin infections and lesions, such as may be present in a shingles outbreak.

In some aspects, the homeopathic composition may include Citrus aurantium dulcis (orange) peel oil, which may be extracted from the peels of both sweet and sour oranges by cold compression. Orange oil extract may have inflammatory qualities, which may detoxify congested skin and soothe dry or irritated skin, such as may occur in shingles, dermatitis, and acne outbreaks.

In some embodiments, the homeopathic composition may include Coenzyme A, which may be adapted from pantothenic acid and adenosine triphosphate. Biologically speaking, this coenzyme plays a vital role in the synthesis and oxidation of fatty acids. Coenzyme A may function as a skin conditioner, emollient, and solvent, and in some aspects, coenzyme A may stimulate collagen production.

In some aspects, the homeopathic composition may include Dehydroacetic Acid. As a mild acid, dehydroacetic acid may work with benzyl alcohol as a preservative and antimicrobial to provide a broad protection from contamination. Dehydroacetic acid may function as a fungicide and/or bactericide, which, in some embodiments, may be used in personal care products at a maximum concentration of 0.6%.

In some embodiments, the homeopathic composition may include Ginkgo Biloba Leaf Extract, which may be a potent antioxidant that may improve blood flow. Improved blood flow may allow for more effective healing of skin lesions associated with shingles.

In some aspects, the homeopathic composition may include Gluconolactone, which is an ester of gluconic acid and is composed of multiple water-attracting hydroxyl groups. Gluconolactone may hydrate skin and enhance the degree of moisturization of the homeopathic composition. Gluconolactone is a polyhydroxy acid (PHA) that is capable of chelating metals and may also function by scavenging free radicals. In some embodiments, gluconolactone may facilitate healing of outbreaks associated with shingles.

In some embodiments, the homeopathic composition may include Hydrolyzed Lupine Protein, which is a hydrolyzed protein from the seeds of the lupine plant (lupinus). The seeds are high in proteins and contain 35-45% of all the essential amino acids. The hydrolyzed lupine protein may have a restructuring and regenerating effect on skin. Lupine peptides are a blend of penta- and hexa-peptides derived from lupin flower protein that may function as an MMP (matrix metalloprotease) inhibitor, which may prevent MMP activity following exposure of the skin to sunlight. Accordingly, the lupine peptides blend may prevent collagen and elastin (the main proteins making up connective tissue) breakdown catalyzed by MMP activity, which may facilitate renewal of the skin's outer layer, which may stimulate the production of collagen & elastin. In some aspects, the hydrolyzed lupine protein may reduce scarring that may be caused by lesions associated with shingles.

In some aspects, the homeopathic composition may include Hydroxypropyl tetrahydropyrantriol, which is a sugar-protein hybrid made from xylose, a sugar found abundantly in beech trees. As a series of amino acids, Hydroxypropyl tetrahydropyrantriol is small enough to penetrate the skin, which may allow for deeper and more effective penetration of the homeopathic composition. Hydroxypropyl tetrahydropyrantriol stimulates the production of glycosaminoglycan's (GAGs), or mucopolysaccharides. GAGs, which are an important component of connective tissue and may increase production on an extracellular matrix. In some embodiments, hydroxypropyl tetrahydropyrantriol may facilitate healing of the skin, such as around acupuncture points . . . such as on and around lesions associated with shingles.

The topically applied composition can be in the form of a lotion, cream, gel, or salve. It has been found that a synergistic therapeutically effective amount of the combination of a vasodilator, a stimulator of lymphatic activity and a mobilizer of white blood cell activity that can be topically applied at the site of pain and numbness is effective to provide relief from the symptom of the disease causing the problems. That is, for use in connection with CRPS diseases such as fibromyalgia, diabetic neuropathy, toxic neuropathy and the like.

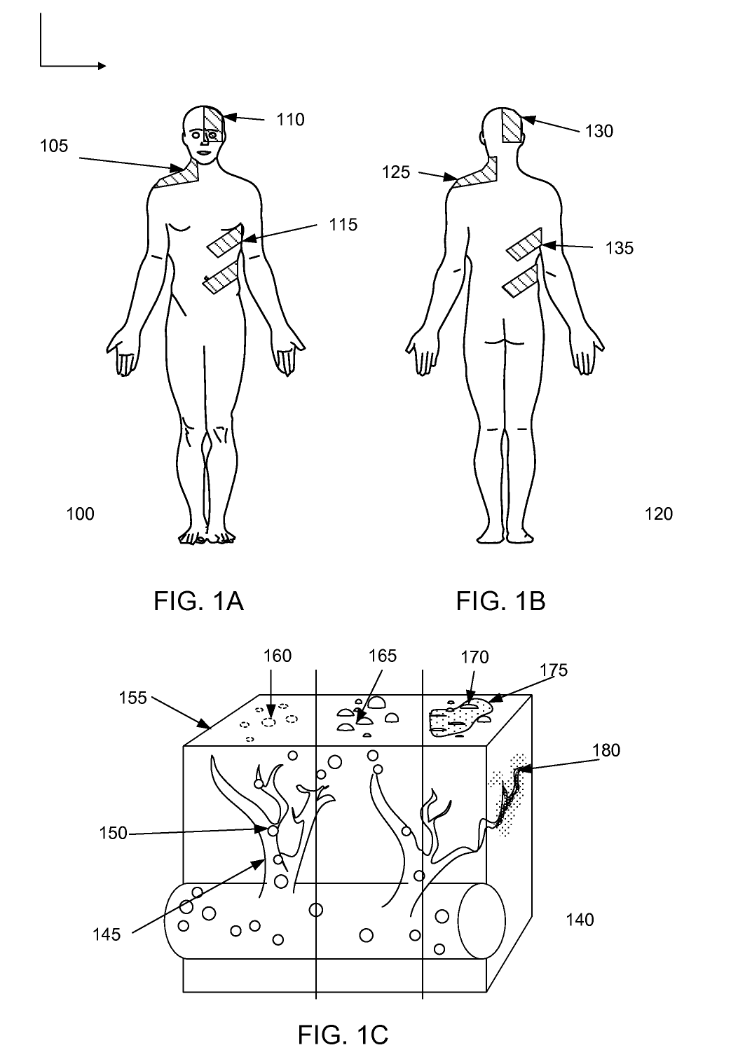

Referring now to FIG. 1A, an anterior view 100 of a human body, wherein common shingles sites are highlighted, is illustrated. Referring now to FIG. 1B, a posterior view 120 of a human body, wherein common shingles sites are highlighted, is illustrated. Shingles outbreaks may commonly occur in the cranial region 110, 130, and some severe cases may develop around the ocular region. Another common area for a diagnosis of a shingles outbreak may be the shoulder region 105, 125. In some cases, a diagnosis may be based upon identification of a striping pattern of outbreak along a torso region 115, 135.

Referring now to FIG. 1C, a progression of symptoms on a cross section of skin 140 with a subcutaneous nerve fiber 145 is illustrated. Such progression of symptoms may support a diagnosis of shingles or other viral driven neuropathy. In the dormant stage, a virus 150 may remain in the nerve fiber 145. The diagnosis of a first stage may include indications of a reactivation state wherein the virus 150 may cause pain 160, fever, or other symptoms. A diagnosis of a subsequent stage may be based upon an outbreak of blisters 165 on the patient's skin surface 155, such as in the regions illustrated in FIGS. 1A-1B. Diagnosis of another stage may be based upon crusting of popped blisters 170 and skin discoloration 175. In some severe cases, the outbreak may cause nerve ending damage 180.

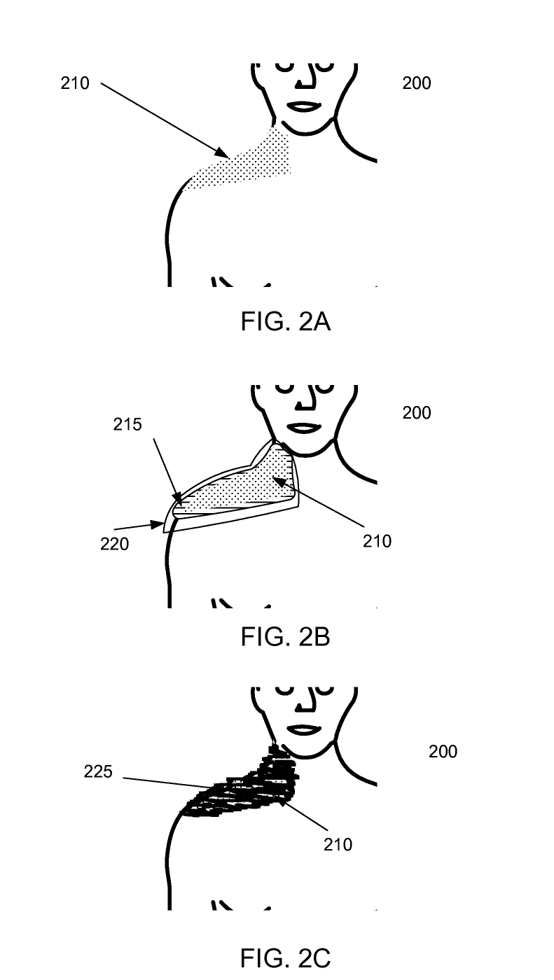

Referring now to FIG. 2A, an exemplary outbreak of an infected user 200 is illustrated, wherein a rash 210 may present on a shoulder region of the user and identified via visual inspection and symptom analysis as one or more manifestations of a virally driven neuropathy.

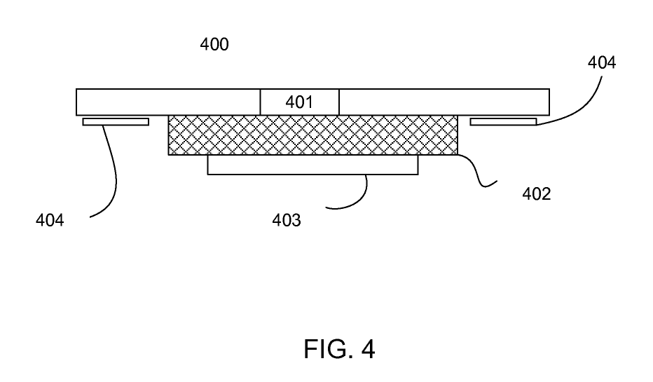

Referring now to FIG. 2B, an exemplary application of the homeopathic composition 215 on an infected user is illustrated, wherein the application includes placing a bandaging material 220 on or proximate to the rash 210. As describer more fully below in conjunction with FIG. 4, in some embodiments, the homeopathic composition application may be combined with a bandage or compress. For example, the bandage may include a portion at least partially saturated with the homeopathic composition, wherein pressure may release the homeopathic composition onto the rash.

In some aspects, the homeopathic composition may be purchased over the counter and administered by the user. In some examples, the adverse drug interactions may be nominal, which may allow the user to manage the topical therapy without requiring a clinician or doctor, who may be managing other therapies associated with treating shingles symptoms.

Referring now to FIG. 2C, an alternate exemplary application of the homeopathic composition 225 on an infected user 200 is illustrated, wherein the homeopathic composition 225 may be manually applied to an affected region. In some aspects, the homeopathic composition may include ingredients that may have a low risk of skin irritation, which may be exacerbated during the acute eruptive phase. In some such examples, the ingredients may be mild enough to allow a user to apply the topical composition near or on lesions, which may be the source or the epicenter of pain symptoms during the second phase.

In some embodiments of the present invention, a homeopathic composition as described herein may be applied directly to an area affected by rash and sores, wherein an application during contagious phases may alleviate painful symptoms and additionally reduce a likelihood of spreading a causative virus. In some aspects, the homeopathic composition may coat the rash with a hygienic barrier. In some embodiments, the homeopathic composition may act in conjunction with an antiviral medication.

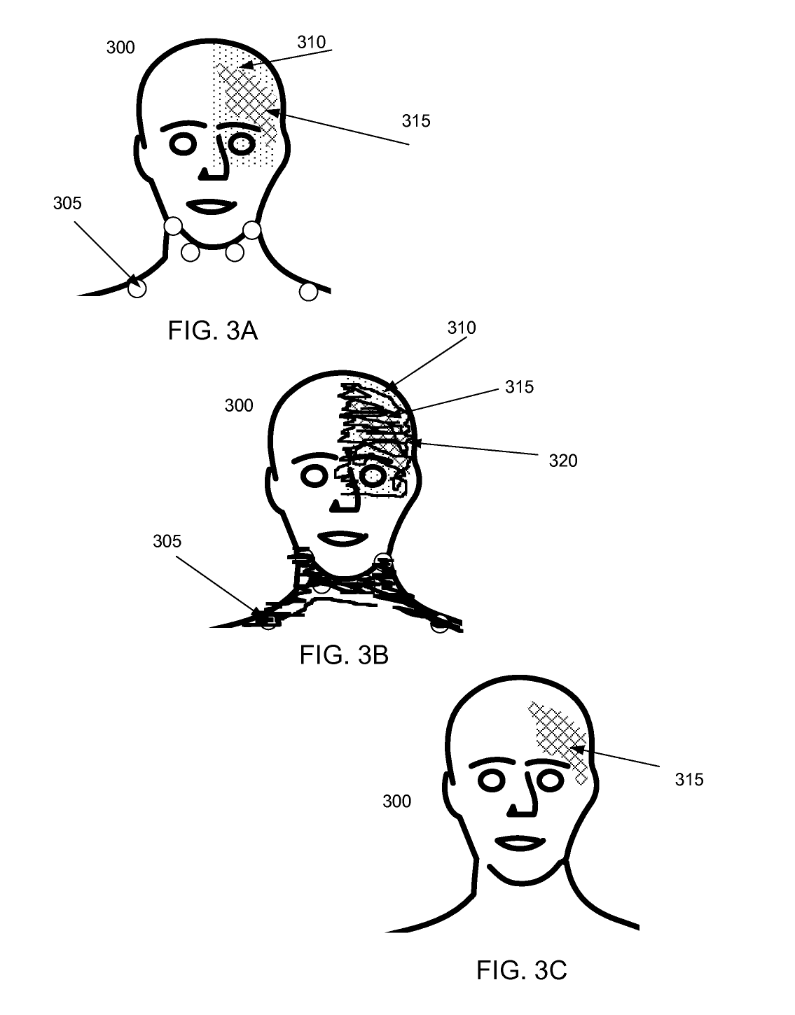

Referring now to FIG. 3A, in some embodiments a diagnosis may be based upon an outbreak of an infected user 300 on a facially located site 310. As illustrated, in some embodiments, an observation of swollen lymph nodes 305 on the neck of the infected user 300 may also be used as an indicator. In some cases, diagnosis of an affected site 310 may include discoloration 315 developed from the rash.

Referring now to FIG. 3B, an exemplary application of the homeopathic composition 320 on the infected user 300 is illustrated. In some embodiments, the homeopathic composition 320 may be applied directly on the affected site 310. In some embodiments, the homeopathic composition 320 may be applied on the lymph nodes 305. Embodiments may also include the homeopathic composition 320 being applied to sensitive or difficult to reach areas of the body. For example, the homeopathic composition 320 may treat craniofacial, facial, and/or lymph node pain.

The application of the homeopathic composition may include a series of stages, wherein the stages may address different aspects of the symptoms. For example, initially, the application on an irritated area may have a cooling effect, which may provide immediate relief. The application may progress into a numbing effect. During rash or crusting stages, the homeopathic composition may have a drying effect, which may provide for one or both of facilitating the healing of the sores and allowing the sores to heal.

Referring now to FIG. 3C, an exemplary infected user 300 after homeopathic composition treatment is illustrated, wherein the homeopathic composition 320 reduces skin discoloration 315 that may be caused by the sores. In some embodiments, the homeopathic composition may reduce changes in skin pigmentation that may be caused by the sores. For example, the homeopathic composition may include a cocoa butter additive conducive to reducing discoloration.

Referring now to FIG. 4, in some embodiments, a treatment bandage 400 may include a substrate 402 dosed with the homeopathic composition 403. The substrate 402 may be comprised of a porous portion impregnated with homeopathic composition 403. The substrate 402 may therefore include one or both of a natural fiber, such as a cotton or other plant based fiber and a synthetic fiber or gel. The substrate 402 may also include a dissolvable starch that may dissolves into the skin. The substrate 402 may be fixedly attached to a sealing layer 401 that generally provides a boundary between the homeopathic composition 403 and an ambient environment. The sealing layer 401 may include a polymer, vinyl, latex, plastic or other material formable to a contour of a human body part.

In some embodiments, the sealing layer may include an adhesive layer 404 for removably attaching the substrate to a body part in a fashion that allows the homeopathic composition 403 to be brought into contact with the skin of a user and maintained in contact with the skin. The sealing layer allows that homeopathic composition 403 to remain relatively undisturbed by an ambient environment.

### ILLUSTRATIVE EXAMPLES

Illustrative examples of homeopathic composition formulas are listed and described below. These are exemplary only and should not be considered as limiting.

### Example 1

### A Homeopathic Gel Prepared by Admixing the Following Ingredients

| Ingredient | Wt. % |
|---|---|
| Carbomer 940 | 2.10 |
| Xantham gum | 0.15 |
| Propylene glycol | 51.94 |
| Dipropylene glycol | 10.00 |
| Ethoxydiglycol | 15.00 |
| Dimethylisosorbide | 10.00 |
| Aloe Vera gel | 2.00 |
| Surfactant | 0.05 |
| Amica Montana | 2.50 |
| Belladooa | 2.50 |
| Rhus toxicodendron | 2.00 |
| Aescuius hippocastanum | 1.76 |

Although the specific activity of each of either plants or herbs have been recognized, it has been surprisingly found that the combination as now claimed has been found to produce the desired effect. The composition is applied to the limb having pain 1-8 times per day.

### Example 2

A creme was formed by admixing the following ingredients:

| Ingredient | | |
|---|---|---|
| Aesculus hippocastanum extract | 6 | mcg/g |
| Amica Montana extract | 6 | mcg/g |
| L-arginine | 6 | mcg/g |
| Echinacea augustfolia extract | 6 | mcg/g |
| Rhus toxicondendron extract | 6 | mcglg |
| Ruta graveoleus extract | 6 | mcg/g |
| Graphites | 6 | mcg/g |
| Crotalus horridus extract | 0.08 | mcg/g |
| Heloderma horridum extract | 0.08 | mcg/g |
| Lachesis extract | 0.08 | mcg/g |
| Naja extract | 0.08 | mcg/g |
| Isopropyl myristate | 30 | mg/g |
| Brij 72 | 2 | mg/g |
| Brij 72IS | 2 | mg/g |
| Water | 83 | mg/g |
| Methyl paraben | 0.2 | mg/g |
| Propyl paraben | 20 | mcg/g |
| Glycerine | 3 | mg/g |
| 20% NaOH | 3 | mg/g |
| Dowcil 200 | 50 | mcg/g |
| Akodel 112 | 2 | mg/g |

The cream may be used to treat cracked skin on feet or anhidrosis, which is associated with CRPS.

### Example 3

A lotion is prepared by admixing the following ingredients:

| Ingredient | Wt. % |
|---|---|
| Ginger | 1.10 |
| Propylene Glycol Stearate | 6.50 |
| Isocetyl alcohol | 5.00 |
| PEG-100 Stearate | 1.20 |
| Water | 69.90 |
| Echinacea augustfolia extract | 3.00 |
| Methyl paraben | 0.20 |
| Propylene glycol | 12.00 |
| Sorbitan palmitate | 0.60 |
| Arnica Montana | 3.00 |
| Aesculus hippocastanum extract | 2.00 |
| Barberry | 1.00 |
| Mate extract | 0.50 |

If desired, 3% by weight capsaicin can be added. The lotion can be used to treat a user suffering from fibromyalgia.

### Example 4

### Preparation of a Gel

| Ingredient | Wt. % |
|---|---|
| Arnica Montana | 5.00 |
| Primrose oil | 3.00 |
| Arginine base (10% solution) | 5.00 |
| (Ajinomoto) | |
| Carbopol 940 | 0.40 |
| Butylene glycol | 6.50 |
| Echinacea augustfolia | 3.00 |
| Chamomile glycolic extract | 3.00 |
| Crotalus horridus venom | 0.50 |
| Preservative | 0.10 |
| Fragrance | 0.10 |
| Deionized water | g.s. |

To 20 ml of water with stirring is added the Carbopol 940. The mixture is stirred until hydration is complete and then butylene glycol is added. The arginine base is then added to the mixture. The remaining ingredients are mixed together and added to the first mixture. The mixing is continued until uniform. The composition can be applied 1-8 times daily to reduce the pain resulting from CRPS.

### Example 5

A gel is prepared by admixing the following ingredients:

| Ingredient | Wt. % |
|---|---|
| Propylene Glycol | 43 |
| Polyacrylic acid | 2.1 |
| Dipropylene Glycol | 16 |
| Xantbam Gum | 0.15 |
| Etboxydiglycol | 15 |
| Dimetbylisosorbide | 10 |
| Ascorbic Acid | 2 |
| Chloroxylenol | 0.2 |
| Linoleamidopropyl PG- | 1.5 |
| diammonium chloride phosphate | |
| Glyceretb 4.5 Lactate | 2 |
| Naja venom extract | 2 |
| Echinacea augustfolia | 2 |
| Octoxynol-9 | 0.5 |
| Primrose Oil | 2 |
| Cocamidopropyl PG-dimon | 1 |
| chloride phosphate | |
| Water | 6 |
| Ginger | 0.44 |

Ingredients 1 and 2 are mixed to disperse and form a gel. About 80% of ingredient 3 is mixed with ingredient 4, added to the gel slightly heated with admixture. The balance of 3 is mixed with ingredients 5-17 and added to the gel at 38 degrees. After mixing, the pH is adjusted to about 4 and then the gel is brought to room temperature. Arnica Montana may also be added to have a plurality of dual acting ingredients. Capsaicin together with the ginger can be used to relieve pain.

### CONCLUSION

A number of embodiments of the present invention have been described. While this specification contains many specific implementation details, there should not be construed as limitations on the scope of any inventions or of what may be claimed, but rather as descriptions of features specific to particular embodiments of the present invention.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

While the invention has been described in conjunction with specific embodiments, it is evident that many alternatives, modifications and variations will be apparent to those skilled in the art in light of the foregoing description. Accordingly, this description is intended to embrace all such alternatives, modifications, and variations as fall within its spirit and scope.

Although shown and described in what is believed to be the most practical and preferred embodiments, it may be apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims

What is claimed is:

1. A method for treating shingles, the method comprising the steps of:
identifying a situs of a shingles condition on human skin; and
applying a topical composition onto the situs of the shingles condition on human skin, wherein the composition comprises an effective amount of a combination of herbs or extracts of Arnica Montana, Rhus toxicodendron and Aesculus hippocastanum mixed with an effective amount of belladona, in a pharmaceutically acceptable carrier to alleviate pain associated with the shingles condition.

2. The method of claim 1 wherein the composition comprises a gel.

3. The method of claim 1 wherein the composition comprises a lotion.

4. The method of claim 1 wherein the composition comprises a cream.

5. The method of claim 1 additionally comprising the steps of observing pain and fever in conjunction with the identification of the situs of the shingles condition and topically applying the topical composition onto the situs of shingles.

6. The method of claim 5 additionally comprising the step of observing blisters on an area of skin in conjunction with the identification of the situs of the shingles condition and topically applying the topical composition onto the blisters.

7. The method of claim 6 additionally comprising the step of observing crusting on the blisters on the area of skin in conjunction with the identification of the situs of the shingles condition and topically applying the topical composition onto crusting blisters.

8. The method of claim 7 additionally comprising the step of applying treatment bandage over the situs of shingles on the human skin wherein the treatment bandage comprises a porous portion impregnated with the topical composition.

9. The method of claim 8 wherein the treatment bandage applied to the situs of shingles additionally comprises:
a. a sealing layer formable to a contour of the situs manifesting shingles;
b. a substrate fixedly attached to the sealing layer; and
c. a treating composition one or both of within and on the surface of the substrate fixedly attached to the sealing layer, the treating composition comprising an effective amount of a combination of herbs or extracts of Arnica Montana, Rhus toxicodendron and Aesculus hippocastanum mixed with an effective amount of belladona, in a pharmaceutically acceptable carrier.

10. The method of claim 5 additionally comprising the step of observing discoloration on an area of skin in conjunction with the identification of the situs of the shingles condition and topically applying the topical composition onto the area of discoloration.

11. A treatment bandage for alleviating discomfort associated with shingles manifesting situs on human skin, the treatment bandage comprising:
a first sealing layer formable to a contour of the situs manifesting shingles;
a substrate fixedly attached to the first sealing layer; and
a treating composition on a first surface of the substrate fixedly attached to the first sealing layer, the treating composition comprising an effective amount of a combination of herbs or extracts of Arnica Montana, Rhus toxicodendron and Aesculus hippocastanum mixed with an effective amount of belladona, in a pharmaceutically acceptable carrier.

12. The treatment bandage of claim 11 wherein the treating composition comprises a gel.

13. The treatment bandage of claim 11 wherein the treating composition comprises a lotion.

14. The treatment bandage of claim 11 wherein the treating composition comprises a cream.

15. The treatment bandage of claim 14 additionally comprising a second sealing layer for removably attaching the substrate and treating composition in a position proximate and in contact with the situs of shingles on the human skin.
